SEQUENCE LISTING (2 sequences)

SEQ ID NO: 1 - DNA, 21 bp, Artificial Sequence
  synthetic
gcagcacgacuucuucaagtt

SEQ ID NO: 2 - DNA, 21 bp, Artificial Sequence
  synthetic
cuugaagaagucgugcugctt

(19) United States
(12) Patent Application Publication
(10) Pub. No.: US 2004/0214198 A1
(43) Pub. Date: Oct. 28, 2004

(54) Allele-targeted RNA interference

(75) Inventor: Tariq M. Rana, Shrewsbury, MA (US)

Correspondence Address: LAHIVE & COCKFIELD, LLP., 28 STATE STREET, BOSTON, MA, 02109, US

(73) Assignee: UNIVERSITY OF MASSACHUSETTS, Worcester, MA

(21) Appl. No.: 10/715,229

(22) Filed: Nov. 17, 2003

Related U.S. Application Data
(60) Provisional application No. 60/426,982, filed on Nov. 15, 2002.
(60) Provisional application No. 60/430,517, filed on Nov. 26, 2002.
(60) Provisional application No. 60/458,051, filed on Mar. 26, 2003.

(51) Int. Cl. C12Q 1/68; C07H 21/02; A61K 48/00

(52) U.S. Cl. 435/6; 435/375; 514/44; 536/23.1

(57) ABSTRACT

The present invention provides siRNAs with modified bases in the antisense strand, e.g., 5-Iodo-Uridine (U(5I)), 5-Bromo-uridine (U(5Br)), or DAP, and methods for using the modified siRNAs to selectively down-regulate the expression of a mutant allele, even when the mutant mRNA differs from wild-type by only a single nucleotide.

### RELATED APPLICATIONS

This patent application claims the benefit of U.S. Provisional Patent Application Serial No. 60/430,517, entitled “Allele-Targeted RNA Interference”, filed Nov. 26, 2002; U.S. Provisional Patent Application Serial No. 60/426,982, entitled “In Vivo Gene Silencing by Chemically Modified and Stable siRNA, filed Nov. 15, 2002; U.S. Provisional Patent Application Serial No. 60/458,051, entitled “In Vivo Gene Silencing by Chemically Modified and Stable siRNA, filed Mar. 26, 2003. The entire contents of the above-referenced provisional patent applications are incorporated herein by this reference.

### BACKGROUND

Diseases caused by dominant, gain-of-function gene mutations develop in heterozygotes bearing one mutant and one wild type copy of the gene. Some diseases of this class are neurodegenerative diseases, including Alzheimer's disease, Huntington's disease, Parkinson's disease, and amyotrophic lateral sclerosis (ALS; “Lou Gehrig's disease”)(1). In these diseases, the exact pathways whereby the mutant proteins cause cell degeneration are not clear, but the origin of the cellular toxicity is known to be the mutant protein.

### SUMMARY

The invention is based in part on the discovery that small interfering RNAs (siRNAs) can be modified to selectively inhibit expression of a mutant allele, e.g., an allele with a single base difference, while preserving expression of the wild-type.

Accordingly, in one aspect, the present invention includes siRNA molecules, e.g., comprising one or more modified bases. In one embodiment, the invention features a small interfering RNA (siRNA) comprising at least one modified base, wherein the modified base is capable of enhancing single nucleotide discrimination between a first target having 1, 2, 3 or more mutations relative to a second target.

In one embodiment, the invention features a small interfering RNA (siRNA) capable of single nucleotide discrimination between a first and second allele, the first allele having 1, 2, 3 or more mutations relative to the second allele, wherein the siRNA comprises at least one modified base capable of enhancing binding interactions between the siRNA and mRNA encoded by the first allele when compared with binding interactions between the siRNA and mRNA encoded by the second allele.

In one embodiment, the invention features a small interfering RNA (siRNA) comprising a sense strand and an antisense strand, wherein the sense strand comprises a sequence homologous to a region of a mutant allele encoding a gain-of-function mutant protein, said region comprising one or more point mutations, and wherein the antisense strand comprises a sequence comprising one or more modified bases positioned opposite the point mutations, such that the siRNA directs allele-specific cleavage of a mRNA encoded by the mutant allele.

In preferred embodiments, the modified base is selected from the group consisting of 5-bromo-uridine, 5-bromo-cytidine, 5-iodo-uridine, 5-iodo-cytidine, 2-amino-purine, 2-amino-allyl-purine, 6-amino-purine, 6-amino-allyl-purine, 2,6-diaminopurine and 6-amino-8-bromo-purine. In an exemplary embodiment, the modified base is 5-bromo-uridine or 5-iodo-uridine and, e.g., the point mutation is an adenine. In another exemplary embodiment, the modified base is 2,6-diaminopurine and, e.g., the point mutation is a thymine.

In embodiments of the invention, the siRNA is between about 10 and 50 residues in length, between about 15 and 45 residues in length, between about 20 and 40 residues in length, or between about 18-25 residues in length.

In one aspect, the present invention features a method of selectively targeting in a cell a first allele having 1, 2, 3 or more mutations relative to a second allele, involving contacting the cell with an siRNA of the invention having a sequence specific for the first allele, such that the first allele is selectively targeted.

In one aspect, the invention features a method of inhibiting expression of a target allele in a cell comprising at least two different alleles of a gene, the method comprising introducing into the cell an siRNA of the invention having a sequence specific for the target allele, said siRNA being introduced in an amount sufficient for degradation of a mRNA encoded by the target allele to occur, thereby inhibiting expression of the target allele. The cell can be isolated, or in an animal, e.g., a mammal, e.g., a human being. In a related aspect, the invention provides a method of activating allele-specific RNA interference (RNAi) in an organism comprising at least two different alleles of a gene, the method comprising administering to the organism an siRNA of the invention having a sequence specific for the target allele, said siRNA being administered in an amount sufficient for degradation of the target allele mRNA to occur, thereby activating allele-specific RNAi in the organism. In one embodiment, the expression is inhibited by at least 10%.

In one aspect, the invention provides a host cell, e.g., a mammalian cell, and preferably a human cell, comprising the siRNAi of the invention. In one embodiment the cell is an embryonic stem cell.

In one aspect, the invention provides an organism obtained by the methods of the invention.

In another aspect, the invention provides a therapeutic composition comprising a siRNA of the invention and a pharmaceutically acceptable carrier.

In yet another aspect, the invention features a method of treating a subject having a disease or disorder correlated with the presence of a dominant gain-of-function mutant allele, the method comprising administering to the subject an siRNA of the invention having a sequence specific for the mutant allele, said siRNA being administered in an amount sufficient for degradation of a mRNA encoded by the mutant allele to occur, thereby treating the subject.

In one embodiment of the invention, the mutant allele comprises one or more point mutations. In various embodiments of the invention, the target allele is correlated with a disease or disorder associated with a dominant gain-of-function mutation. In one embodiment, the siRNA is targeted to the gain-of-function mutation. Preferably, the disease or disorder is chosen from the group consisting of amyotrophic lateral sclerosis, Huntington's disease, Alzheimer's disease, and Parkinson's disease. In a preferred embodiment, the disease is amyotrophic lateral sclerosis. In a further embodiment, the allele is SOD1.

So that the invention may be more readily understood, certain terms are first defined.

“Allele specific inhibition of expression” refers to the ability to significantly inhibit expression of one allele of a gene over another, e.g., when both alleles are present in the same cell. For example, the alleles can differ by one, two, three, or more nucleotides. In some cases, one allele is associated with disease causation, e.g., a disease correlated to a dominant gain-of-function mutation.

The term “allele” as used herein, refers to one of two alternate forms of a gene that can have the same locus on homologous chromosomes. Two different alleles may be responsible for alternative traits, e.g., one allele can be dominant over the other.

The term “nucleoside” refers to a molecule having a purine or pyrimidine base covalently linked to a ribose or deoxyribose sugar. Exemplary nucleosides include adenosine, guanosine, cytidine, uridine and thymidine. The term “nucleotide” refers to a nucleoside having one or more phosphate groups joined in ester linkages to the sugar moiety. Exemplary nucleotides include nucleoside monophosphates, diphosphates and triphosphates. The terms “polynucleotide” and “nucleic acid molecule” are used interchangeably herein and refer to a polymer of nucleotides joined together by a phosphodiester linkage between 5′ and 3′ carbon atoms.

The term “RNA” or “RNA molecule” or “ribonucleic acid molecule” refers to a polymer of ribonucleotides. The term “DNA” or “DNA molecule” or deoxyribonucleic acid molecule” refers to a polymer of deoxyribonucleotides. DNA and RNA can be synthesized naturally (e.g., by DNA replication or transcription of DNA, respectively). RNA can be post-transcriptionally modified. DNA and RNA can also be chemically synthesized. DNA and RNA can be single-stranded (i.e., ssRNA and ssDNA, respectively) or multi-stranded (e.g., double stranded, i.e., dsRNA and dsDNA, respectively). “mRNA” or “messenger RNA” is single-stranded RNA that specifies the amino acid sequence of one or more polypeptide chains. This information is translated during protein synthesis when ribosomes bind to the mRNA.

As used herein, the term “small interfering RNA” (“siRNA”) (also referred to in the art as “short interfering RNAs”) refers to an RNA (or RNA analog) comprising between about 10-50 nucleotides (or nucleotide analogs) which is capable of directing or mediating RNA interference.

The term “nucleotide analog”, also referred to herein as an “altered nucleotide” or “modified nucleotide” refers to a non-standard nucleotide, including non-naturally occurring ribonucleotides or deoxyribonucleotides. Preferred nucleotide analogs are modified at any position so as to alter certain chemical properties of the nucleotide yet retain the ability of the nucleotide analog to perform its intended function.

The term “oligonucleotide” refers to a short polymer of nucleotides and/or nucleotide analogs. The term “RNA analog” refers to an polynucleotide (e.g., a chemically synthesized polynucleotide) having at least one altered or modified nucleotide as compared to a corresponding unaltered or unmodified RNA but retaining the same or similar nature or function as the corresponding unaltered or unmodified RNA. As discussed above, the oligonucleotides may be linked with linkages which result in a lower rate of hydrolysis of the RNA analog as compared to an RNA molecule with phosphodiester linkages. For example, the nucleotides of the analog may comprise methylenediol, ethylene diol, oxymethylthio, oxyethylthio, oxycarbonyloxy, phosphorodiamidate, phophoroamidate, and/or phosphorothioate linkages. Exemplary RNA analogues include sugar- and/or backbone-modified ribonucleotides and/or deoxyribonucleotides. Such alterations or modifications can further include addition of non-nucleotide material, such as to the end(s) of the RNA or internally (at one or more nucleotides of the RNA). An RNA analog need only be sufficiently similar to natural such RNA that it has the ability to mediate (mediates) RNA interference.

As used herein, the term “RNA interference” (“RNAi”) refers to a selective (i.e., target-specific) degradation of RNA. RNAi occurs in cells naturally to remove foreign RNAs (e.g., viral RNAs). Natural RNAi proceeds via fragments cleaved from free dsRNA which direct the degradative mechanism to other similar RNA sequences. Alternatively, RNAi can be initiated by the hand of man, for example, to silence the expression of target genes. RNAi can be initiated intracellularly or in, for example, cell extracts.

A siRNA having a “sequence sufficiently complementary to a target mRNA sequence to direct target-specific RNA interference (RNAi)” means that the siRNA has a sequence sufficient to trigger the destruction of the target mRNA by the RNAi machinery or process.

A gene “involved” in a disorder includes a gene, the normal or aberrant expression or function of which effects or causes a disease or disorder or at least one symptom of said disease or disorder

A “target gene” is a gene whose expression is to be selectively inhibited or “silenced.” A “target allele” is an allele whose expression is to be selectively inhibited or “silenced.” This silencing is achieved by cleaving the mRNA of the target gene or target allele by an siRNA. One strand of the siRNA is an antisense strand that is complementary, e.g., fully complementary, to a section of about 16 to 30 or more nucleotides of the mRNA of the target gene or target allele.

An “isolated nucleic acid molecule or sequence” is a nucleic acid molecule or sequence that is not immediately contiguous with both of the coding sequences with which it is immediately contiguous (one on the 5′ end and one on the 3′ end) in the naturally occurring genome of the organism from which it is derived. The term therefore includes, for example, a recombinant DNA or RNA that is incorporated into a vector; into an autonomously replicating plasmid or virus; or into the genomic DNA of a prokaryote or eukaryote, or which exists as a separate molecule (e.g., a cDNA or a genomic DNA fragment produced by PCR or restriction endonuclease treatment) independent of other sequences. It also includes a recombinant DNA that is part of a hybrid gene encoding an additional polypeptide sequence.

The term “engineered,” as in an engineered RNA precursor, or an engineered nucleic acid molecule, indicates that the precursor or molecule is not found in nature, in that all or a portion of the nucleic acid sequence of the precursor or molecule is created or selected by man. Once created or selected, the sequence can be replicated, translated, transcribed, or otherwise processed by mechanisms within a cell. Thus, an RNA precursor produced within a cell from a transgene that includes an engineered nucleic acid molecule is an engineered RNA precursor.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1A is a drawing of the structure of 5-iodo-uridine U(5I).

FIG. 1B is a bar graph illustrating the effect of U(5I) modified siRNA on expression of enhanced green fluorescent protein (EGFP) in HeLa cells.

FIG. 2A is a drawing of the structure of 2,6-diaminopurine (DAP).

FIG. 2B is a bar graph illustrating the effect of DAP modified siRNA on expression of EGFP in HeLa cells.

FIG. 3A is a drawing of the structure of 5-bromo-uridine U(5Br).

FIG. 3B is a bar graph illustrating the effect of U(5Br) modified siRNA on expression of enhanced green fluorescent protein (EGFP) in HeLa cells.

FIG. 4 is a representation of the sequences of the sense [SEQ ID NO.: 1] and antisense [SEQ ID NO.: 2] strands of an EGFP siRNA. Lines below the antisense strand indicate adenine bases modified with DAP. Triangles indicate uracils modified to U(5I) or U(5Br).

### DETAILED DESCRIPTION

The present invention is based in part on the discovery that siRNAs with modified bases in the antisense strand, e.g., 5-iodo-uridine (U(5I)), 5-bromo-uridine (U(5Br)), or DAP, can be used to selectively down-regulate the expression of an allele (e.g., a mutant), even when the allelic mRNA differs from a second allele (e.g., wild-type) by only a single nucleotide, as is the case with certain mutations, e.g., mutations of SOD1 correlated with ALS. These methods are applicable to the treatment of diseases that are caused by dominant, gain-of-function type of gene mutations, including, but not limited to, ALS. The siRNAs of the present invention are capable of single nucleotide discrimination and selectively down-regulating expression of their target alleles.

Sequence-selective, post-transcriptional inactivation of gene expression can be achieved in a wide variety of eukaryotes by introducing double-stranded RNA corresponding to the target gene, a phenomenon termed RNAi (2-4). RNAi methodology has been extended to cultured mammalian cells (9-10). This approach takes advantage of the discovery that siRNA can trigger the degradation of mRNA corresponding to the siRNA sequence. It is demonstrated herein that modified siRNA duplexes can be used to preferentially block the expression of a mutant allele, while preserving the expression of a co-expressed wild type allele.

The present methods allow for the selective silencing of a selected target allele, while allowing another allele to remain unaffected, even where the two alleles differ by only a single amino acid. Within the scope of the present method is the use of modified siRNAs to selectively target one allele. Where the mutation results in the replacement of a base in the target mRNA with an adenine, siRNAs modified with U(5Br) or U(5I) in the antisense strand are generally used. Where the mutation results in the replacement of a base in the target RNA with a uracil (thymine in the DNA), siRNAs modified with DAP in the antisense strand are generally used. Without wishing to be bound by theory, it is believed that the favorable binding interactions between the mutant/target mRNA and the modified siRNA and the less favorable binding interactions between the wild-type mRNA and the modified siRNA cause the modified siRNAs to bind preferentially to the mutant target mRNA, leaving the wild-type mRNA untouched.

### I. RNA Interference (RNAi)

RNAi is a remarkably efficient process whereby double-stranded RNA (dsRNA) induces the sequence-specific degradation of homologous mRNA in animals and plant cells (Hutvagner and Zamore (2002), Curr. Opin. Genet. Dev., 12, 225-232; Sharp (2001), Genes Dev., 15, 485-490). In mammalian cells, RNAi can be triggered by 21-nucleotide (nt) duplexes of small interfering RNA (siRNA) (Chiu et al. (2002), Mol. Cell., 10, 549-561; Elbashir et al. (2001), Nature, 411, 494-498), or by micro-RNAs (miRNA), functional small-hairpin RNA (shRNA), or other dsRNAs which are expressed in vivo using DNA templates with RNA polymerase III promoters (Zeng et al. (2002), Mol. Cell, 9, 1327-1333; Paddison et al. (2002), Genes Dev., 16, 948-958; Lee et al. (2002), Nature Biotechnol., 20, 500-505; Paul et al. (2002), Nature Biotechnol., 20, 505-508; Tuschl, T. (2002), Nature Biotechnol., 20, 440-448; Yu et al. (2002), Proc. Natl. Acad. Sci. USA, 99(9), 6047-6052; McManus et al. (2002), RNA, 8, 842-850; Sui et al. (2002), Proc. Natl. Acad. Sci. USA, 99(6), 5515-5520.)

### II. siRNA Molecules

The present invention features “small interfering RNA molecules” (“siRNA molecules” or “siRNA”) and methods (e.g., therapeutic methods) for using said siRNA molecules. The siRNA molecules of the invention comprise dsRNA molecules having one or more modified bases in the antisense strand, e.g., 5-iodo-uridine (U(5I)), 5-bromo-uridine (U(5Br)), and/or 2,6-diaminopurine (DAP). The siRNAs can comprise 16-30, e.g., 16, 17, 18, 19, 20, 21, 22, 23, 24, 25, 26, 27, 28, 29, or 30 nucleotides in each strand, wherein one of the strands is substantially complementary, e.g., at least 80% complementary (or more, e.g., 85%, 90%, 95%, or 100%)(for example, having 3, 2, 1, or 0 mismatched nucleotide(s)), to a target region and the other strand is identical or substantially complementary to the first strand. A target region differs by at least one base pair between the wild type and mutant allele, e.g., a target region comprising a gain-of-function mutation. Preferably, the strands are aligned such that there are at least 1, 2, or 3 bases at the end of the strands which do not align (i.e., for which no complementary bases occur in the opposing strand) such that an overhang of 1, 2 or 3 residues occurs at one or both ends of the duplex when strands are annealed. The siRNA molecules of the invention further have a sequence that is “sufficiently complementary” to a target mRNA sequence to direct target-specific (e.g., allele-specific) RNA interference, as defined herein, i.e., the siRNA has a sequence sufficient to mediate RNAi, e.g., to trigger the destruction of the target mRNA (e.g., mRNA encoding a dominant gain-of-function mutant protein) by the RNAi machinery or process.

The dsRNA molecules of the invention can be chemically synthesized or can be transcribed in vitro from a DNA template or engineered RNA precursor. The siRNAs of the invention generally have one or more modified bases in the antisense strand, e.g., U(5Br), U(5I), and/or DAP. Such modified siRNAs can be synthesized with the modified base.

The featured modified siRNAs of the invention preferably comprise one or more modified nucleobases, wherein the nucleobases are capable of enhancing the specificity of the siRNA, e.g., to a target mutant allele as compared to a corresponding wild-type allele. Such modified nucleobases can be modified pyrimidines and/or purines, e.g., modified uracil, cytosine, adenine or guanine. Thus the modified siRNAs of the invention are modified at positions targeting an allelic mutation, e.g., an allele-specific dominant gain-of-function mutation, (herein also referred to as “siRNAs modified at targeted positions” or “target-modified siRNAs”)

In one embodiment, nucleobase-modified nucleotides useful in the invention comprise a modified pyrimidine, including, but not limited to: ribo-thymidine, 4-thio-uridine, 3-methyl-uridine, 5-bromo-uridine, 5-iodo-uridine, 5-fluoro-uridine, 5-amino-allyl-uridine (e.g., 5-amino-methyl-uridine, 5-amino-ethyl-uridine, 5-amino-propyl-uridine, 5-amino-isopropyl uridine, and the like), 5,6-dihydro-uridine, 3-methyl-cytidine, 5-bromo-cytidine, 5-iodo-cytidine, 5-fluoro-cytidine, 5-amino-allyl-cytidine (e.g., 5-amino-methyl-cytidine, 5-amino-ethyl-cytidine, 5-amino-propyl-cytidine, 5-amino-isopropyl-cytidine, and the like) and 5,6-dihydro-cytidine. Nucleobase-modified nucleotides comprising a modified pyrimidine preferably are 5-bromo-uridine or 5-iodo-uridine.

In another embodiment, nucleobase-modified nucleotides useful in the invention comprise a modified purine, including, but not limited to: 6-thio-guanosine, 2-amino-purine (e.g., 2-amino-adenosine), 2-amino-allyl-purine (e.g., 2-amino-methyl-guanosine, 2-amino-dimethyl-guanosine, 2-amino-ethyl-guanosine, 2-amino-propyl-guanosine, 2-amino-isopropyl-guanosine, 2-amino-methyl-adenosine, 2-amino-dimethyl-adenosine, 2-amino-ethyl-adenosine, 2-amino-propyl-adenosine, and 2-amino-isopropyl-adenosine), 6-amino-purine (e.g., 6-amino-guanosine), 6-amino-allyl-purine (e.g., 6-amino-methyl-adenosine, 6-amino-dimethyl-adenosine, 6-amino-ethyl-adenosine, 6-amino-propyl-adenosine, and 6-amino-isopropyl-adenosine, 6-amino-methyl-guanosine, 6-amino-dimethyl-guanosine, 6-amino-ethyl-guanosine, 6-amino-propyl-guanosine, 6-amino-isopropyl-guanosine) and 2,6-diaminopurine. Nucleobase-modified nucleotides comprising a modified purine are preferably 2,6-diaminopurine. In yet another embodiment, nucleobase-modified nucleotides useful in the invention can comprise a purine modified at two positions, e.g., 6-amino-2-bromo-purine, 6-amino-2-iodo-purine, 6-amino-2-fluoro-purine, 6-amino-8-bromo-purine, 6-amino-8-iodo-purine, 6-amino-8-fluoro-purine, 6-iodo-8-amino-purine, 6-bromo-8-amino-purine, 6-fluoro-8-amino-purine, and the like. A nucleobase-modified nucleotide comprising a purine modified at two positions is preferably 6-amino-8-bromo-purine.

The dsRNA molecules can be designed using any method known in the art, for instance, by using the following protocol:

1. Beginning with an AUG start codon, search for AA dinucleotide sequences; each AA and the 3′ adjacent 16 or more nucleotides are potential siRNA targets. The siRNA should be specific for a target region that differs by at least one base pair between the wild type and mutant allele, e.g., a target region comprising the gain-of-function mutation. In cases where the gain-of-function mutation is associated with one or more other mutations in the same gene, the siRNA can be targeted to any of the mutations. In some cases, the siRNA is targeted to an allelic region that does not comprise a known mutation but does comprise an allelic variation of the wild-type (reference) sequence.

The first strand should be complementary to this sequence, and the other strand is identical or substantially identical to the first strand. In one embodiment, the nucleic acid molecules are selected from a region of the target allele sequence beginning at least 50 to 100 nt downstream of the start codon, e.g., of the sequence of SOD1. Further, siRNAs with lower G/C content (35-55%) may be more active than those with G/C content higher than 55%. Thus in one embodiment, the invention includes nucleic acid molecules having 35-55% G/C content. In addition, the strands of the siRNA can be paired in such a way as to have a 3′ overhang of 1 to 4, e.g., 2, nucleotides. Thus in another embodiment, the nucleic acid molecules can have a 3′ overhang of 2 nucleotides, such as TT. The overhanging nucleotides can be either RNA or DNA.

2. Using any method known in the art, compare the potential targets to the appropriate genome database (human, mouse, rat, etc.) and eliminate from consideration any target sequences with significant homology to other coding sequences. One such method for such sequence homology searches is known as BLAST, which is available at www.ncbi.nlm.nih.gov/BLAST.

3. Select one or more sequences that meet your criteria for evaluation.

Further general information about the design and use of siRNA may be found in “The siRNA User Guide,” available at www.mpibpc.gwdg.de/abteilungen/100/105/sirna.html.

Sequence identity may be determined by sequence comparison and alignment algorithms known in the art. To determine the percent identity of two nucleic acid sequences (or of two amino acid sequences), the sequences are aligned for optimal comparison purposes (e.g., gaps can be introduced in the first sequence or second sequence for optimal alignment). The nucleotides (or amino acid residues) at corresponding nucleotide (or amino acid) positions are then compared. When a position in the first sequence is occupied by the same residue as the corresponding position in the second sequence, then the molecules are identical at that position. The percent identity between the two sequences is a function of the number of identical positions shared by the sequences (i.e., % homology=# of identical positions/total # of positions×100), optionally penalizing the score for the number of gaps introduced and/or length of gaps introduced.

The comparison of sequences and determination of percent identity between two sequences can be accomplished using a mathematical algorithm. In one embodiment, the alignment generated over a certain portion of the sequence aligned having sufficient identity but not over portions having low degree of identity (i.e., a local alignment). A preferred, non-limiting example of a local alignment algorithm utilized for the comparison of sequences is the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the BLAST programs (version 2.0) of Altschul, et al. (1990) J. Mol. Biol. 215:403-10.

In another embodiment, the alignment is optimized by introducing appropriate gaps and percent identity is determined over the length of the aligned sequences (i.e., a gapped alignment). To obtain gapped alignments for comparison purposes, Gapped BLAST can be utilized as described in Altschul et al., (1997) Nucleic Acids Res. 25(17):3389-3402. In another embodiment, the alignment is optimized by introducing appropriate gaps and percent identity is determined over the entire length of the sequences aligned (i.e., a global alignment). A preferred, non-limiting example of a mathematical algorithm utilized for the global comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

Greater than 80% sequence identity, e.g., 81%, 82%, 83%, 84%, 85%, 86%, 87%, 88%, 89%, 90%, 91%, 92%, 93%, 94%, 95%, 96%, 97%, 98%, 99% or even 100% sequence identity, between the siRNA and the portion of the target gene is preferred. Alternatively, the siRNA may be defined functionally as a nucleotide sequence (or oligonucleotide sequence) that is capable of hybridizing with a portion of the target gene transcript (e.g., 400 mM NaCl, 40 mM PIPES pH 6.4, 1 mM EDTA, 50° C. or 70° C. hybridization for 12-16 hours; followed by washing). Additional preferred hybridization conditions include hybridization at 70° C. in 1×SSC or 50° C. in 1×SSC, 50% formamide followed by washing at 70° C. in 0.3×SSC or hybridization at 70° C. in 4×SSC or 50° C. in 4×SSC, 50% formamide followed by washing at 67° C. in 1×SSC. The hybridization temperature for hybrids anticipated to be less than 50 base pairs in length should be 5-10° C. less than the melting temperature (Tm) of the hybrid, where Tm is determined according to the following equations. For hybrids less than 18 base pairs in length, Tm(° C.)=2(# of A+T bases)+4(# of G+C bases). For hybrids between 18 and 49 base pairs in length, Tm(° C.)=81.5+16.6(log10[Na+])+0.41 (%G+C)−(600/N), where N is the number of bases in the hybrid, and [Na+] is the concentration of sodium ions in the hybridization buffer ([Na+] for 1×SSC=0.165 M). Additional examples of stringency conditions for polynucleotide hybridization are provided in Sambrook, J., E. F. Fritsch, and T. Maniatis, 1989, Molecular Cloning: A Laboratory Manual, Cold Spring Harbor Laboratory Press, Cold Spring Harbor, N.Y., chapters 9 and 11, and Current Protocols in Molecular Biology, 1995, F. M. Ausubel et al., eds., John Wiley & Sons, Inc., sections 2.10 and 6.3-6.4, incorporated herein by reference. The length of the identical nucleotide sequences may be at least about 10, 12, 15, 17, 20, 22, 25, 27, 30, 32, 35, 37, 40, 42, 45, 47 or 50 bases.

Negative control siRNAs should have the same nucleotide composition as the selected siRNA, but without significant sequence complementarity to the appropriate genome. Such negative controls can be designed by randomly scrambling the nucleotide sequence of the selected siRNA; a homology search can be performed to ensure that the negative control lacks homology to any other gene in the appropriate genome. In addition, negative control siRNAs can be designed by introducing one or more base mismatches into the sequence.

Preferably, an siRNA molecule of the invention will have a three-dimensional structure resembling A-form RNA helix. More preferably, an siRNA molecule of the invention will have an antisense strand which is capable of adopting an A-form helix when in association with a target RNA (e.g., an mRNA). For this reason, 2′-fluro-modified nucleotides are preferred, as siRNA made with such modified nucleotides adopts an A-form helix confirmation. In particular, it is important that an siRNA be capable of adopting an A-form helix in the portion complementary to the target cleavage site as it has been discovered that the major groove formed by the A-form helix at the cleavage site, and not the RNA itself, is an essential determinant of RNAi.

In some embodiments of the invention, an siRNA molecule also exhibits a relatively low level of toxicity. For example, a concentration of an siRNA molecule that inhibits expression of a targeted sequence has relatively low toxicity when at least 50% of the cells in a culture treated with the siRNA derivative are viable when expression of the targeted sequence is decreased by 50% compared to expression in a cell that is not treated with the siRNA derivative. Low toxicity may be associated with greater cell viability, e.g., at least 60%, 75%, 85%, 90%, 95%, or 100%. Methods of measuring cell viability are known in the art and include trypan blue exclusion.

A. Stabilizing Modifications

The featured modified siRNAs (e.g., siRNAs modified at targeted positions) of the invention may additionally comprise other siRNA modifications known in the art, e.g., siRNA modifications designed such that properties important for in vivo applications, in particular, human therapeutic applications, are improved without compromising the RNAi activity of the siRNA molecules e.g., modifications to increase resistance of the siRNA molecules to nucleases. The featured modified siRNA molecules of the invention (e.g., siRNAs modified at targeted positions) can additionally be modified by the substitution of at least one nucleotide with a modified nucleotide, such that, for example, in vivo stability is enhanced as compared to a corresponding target-modified siRNA, or such that the target efficiency is further enhanced compared to a corresponding target-modified siRNA. Such modifications are also useful to improve uptake of the siRNA by a cell. Preferred modified nucleotides do not effect the ability of the antisense strand to adopt A-form helix conformation when base-pairing with the target mRNA sequence, e.g., an A-form helix conformation comprising a normal major groove when base-pairing with the target mRNA sequence.

The featured siRNA molecules of the invention (e.g., siRNAs modified at targeted positions) can be additionally modified at the 5′ end, 3′ end, 5′ and 3′ end, and/or at internal residues, or any combination thereof. Internal siRNA modifications can be, for example, sugar modifications, nucleobase modifications, backbone modifications, and can contain mismatches, bulges, or crosslinks. Also preferred are 3′ end, 5′ end, or 3′ and 5′ and/or internal modifications, wherein the modifications are, for example, cross linkers, heterofunctional cross linkers, dendrimer, nano-particle, peptides, organic compounds (e.g., fluorescent dyes), and/or photocleavable compounds.

The featured siRNA molecules of the invention (e.g., siRNAs modified at targeted positions) can additionally comprise one or more (e.g., about 1, 2, 3, 4, 5, 6, 7, 8, 9, 10, or more) sugar-modified nucleotides. Sugar-modified nucleotides useful in the invention include, but are not limited to: 2′-fluoro modified ribonucleotide, 2′-OMe modified ribonucleotide, 2′-deoxy ribonucleotide, 2′-amino modified ribonucleotide and 2′-thio modified ribonucleotide. The sugar-modified nucleotide can be, for example, 2′-fluoro-cytidine, 2′-fluoro-uridine, 2′-fluoro-adenosine, 2′-fluoro-guanosine, 2′-amino-cytidine, 2′-amino-uridine, 2′-amino-adenosine, 2′-amino-guanosine or 2′-amino-butyryl-pyrene-uridine. Preferably, a 2′-deoxy ribonucleotide is present within the sense strand and, for example, can be upstream of the cleavage site referencing the antisense strand or downstream of the cleavage site referencing the antisense strand. In a preferred embodiment, the sugar-modified nucleotide is a 2′-fluoro modified ribonucleotide, e.g., in the sense and antisense strands, and preferably, e.g., at every uridine and cytidine. In another preferred embodiment, the sugar-modified nucleotide is a 2′-OMe modified ribonucleotide.

The featured siRNA molecules of the invention (e.g., siRNAs modified at targeted positions) can additionally comprise one or more (e.g., about 1, 2, 3, 4, 5, 6, 7, 8, 9, 1 0, or more) backbone-modified nucleotides, for example, a backbone-modified nucleotide containing a phosphorothioate group. The backbone-modified nucleotide is within the sense strand, antisense strand, or preferably within the sense and antisense strands.

The featured siRNA molecules of the invention (e.g., siRNAs modified at targeted positions) can comprise one or more (e.g., about 1, 2, 3, 4, 5, 6, 7, 8, 9, 10, or more) end modifications. Modification at the 5′ end is preferred in the sense strand, and comprises, for example, a 5′-propylamine group. Modifications to the 3′ OH terminus are in the sense strand, antisense strand, or in the sense and antisense strands. A 3′ end modification comprises, for example, 3′-puromycin, 3′-biotin (e.g., a photocleavable biotin) and the like. In some embodiments, the siRNA derivative has at its 3′ terminus a peptide (e.g., a Tat peptide, hox peptide, or other artificial or natural peptide with cell-penetrating activity), a nanoparticle, a peptidomimetic, organic compounds (e.g., a dye such as a fluorescent dye), or a dendrimer. The siRNA derivative may also be mixed with a delivery agent, e.g., a dendrimer, such as PAMAM as described in U.S. Ser. No. 60/430,525 to Tariq M. Rana, titled “Delivery of siRNAs.” Modifying siRNA derivatives in this way can improve cellular uptake or enhance cellular targeting activities of the resulting siRNA derivative as compared to the corresponding siRNA, are useful for tracing the siRNA derivative in the cell, or improve the stability of the siRNA derivative compared to the corresponding siRNA.

Crosslinking can be employed to alter the pharmacokinetics of the composition, for example, to increase half-life in the body. Thus, the featured siRNA molecules of the invention (e.g., siRNAs modified at targeted positions) can additionally comprise one or more (e.g., about 1, 2, 3, 4, 5, 6, 7, 8, 9, 10, or more) crosslinks, e.g., a crosslink wherein the sense strand is crosslinked to the antisense strand of the siRNA duplex, such as crosslinked siRNA derivatives as described in U.S. Provisional Patent Application No. 60/413,529, which is incorporated herein by reference in its entirety. Crosslinkers useful in the invention are those commonly known in the art, e.g., psoralen, mitomycin C, cisplatin, chloroethylnitrosoureas and the like. Preferably, the crosslink is present downstream of the cleavage site referencing the antisense strand, and more preferably, the crosslink is present at the 5′ end of the sense strand. Alternatively, a 3′ OH terminus of one of the strands can be modified, or the two strands can be crosslinked and modified at the 3′OH terminus.

The featured siRNA molecules of the invention (e.g., siRNAs modified at targeted positions) possess enhanced specificity for a target RNA, e.g., an allele-specific mutation relative to the corresponding wild-type allele, due to the presence of one or more modified nucleobases at positions targeting, e.g., opposite to, the mutation. The siRNA molecules of the invention may have an additional advantage of being able to tolerate one or more (e.g., about 1, 2, 3, 4, 5, 6, 7, 8, 9, 10, or more) mismatches between the antisense strand and target mRNA sequences at positions other than the mutation-targeting position(s). Mismatches between the antisense strand and target mRNA sequence, when present, are preferentially downstream of the cleavage site referencing the antisense strand, e.g., present within 1-6 nucleotides from the 3′ end of the antisense strand. The siRNA molecules of the invention (e.g., siRNAs modified at targeted positions) may also tolerate a bulge, e.g., one or more (e.g., about 1, 2, 3, 4, 5, 6, 7, 8, 9, 10, or more) unpaired bases, in the duplex siRNA, e.g., in the sense strand.

The nucleic acid compositions of the invention can be unconjugated or can be conjugated to another moiety, such as a nanoparticle, to enhance a property of the compositions, e.g., a pharmacokinetic parameter such as absorption, efficacy, bioavailability, and/or half-life. The conjugation can be accomplished by methods known in the art, e.g., using the methods of Lambert et al. (2001), Drug Deliv. Rev., 47(1), 99-112 (describes nucleic acids loaded to polyalkylcyanoacrylate (PACA) nanoparticles); Fattal et al. (1998), J. Control Release, 53(1-3), 137-43 (describes nucleic acids bound to nanoparticles); Schwab et al. (1994), Ann. Oncol., 5 Suppl. 4, 55-8 (describes nucleic acids linked to intercalating agents, hydrophobic groups, polycations or PACA nanoparticles); and Godard et al. (1995), Eur. J. Biochem., 232(2), 404-10 (describes nucleic acids linked to nanoparticles).

The featured siRNA molecules of the invention (e.g., siRNAs modified at targeted positions) can comprise any combination of two or more (e.g., about 2, 3,4, 5, 6, 7, 8, 9, 10, or more) additional siRNA modifications as described herein, e.g., such that in vivo stability is enhanced. For example, a siRNA molecule can comprise a combination of two sugar-modified nucleotides, e.g., 2′-fluoro modified ribonucleotides (e.g., 2′-fluoro uridine or 2′-fluoro cytidine) and 2′-deoxy ribonucleotides (e.g., 2′-deoxy adenosine or 2′-deoxy guanosine). The 2′-deoxy ribonucleotides are preferably in the antisense strand, and, for example, can be upstream of the cleavage site referencing the antisense strand or downstream of the cleavage site referencing the antisense strand. The 2′-fluoro ribonucleotides can be in the sense and antisense strands, and preferably, can be every uridine and cytidine.

The nucleic acid molecules of the present invention can also be labeled using any method known in the art; for instance, the nucleic acid compositions can be labeled with a fluorophore, e.g., Cy3, fluorescein, or rhodamine. The labeling can be carried out using a kit, e.g., the SILENCER™ siRNA labeling kit (Ambion). Additionally, the siRNA can be radiolabeled, e.g., using ³H, ³²P, or other appropriate isotope.

B. Production

RNA may be produced enzymatically or by partial/total organic synthesis, any modified ribonucleotide can be introduced by in vitro enzymatic or organic synthesis. In one embodiment, a siRNA is prepared chemically. Methods of synthesizing RNA molecules are known in the art, in particular, the chemical synthesis methods as de scribed in Verma and Eckstein (1998) Annul Rev. Biochem. 67:99-134. In another embodiment, a siRNA is prepared enzymatically. For example, a ds-siRNA can be prepared by enzymatic processing of a long ds RNA having sufficient complementarity to the desired target mRNA. Processing of long ds RNA can be accomplished in vitro, for example, using appropriate cellular lysates and ds-siRNAs can be subsequently purified by gel electrophoresis or gel filtration. ds-siRNA can then be denatured according to art-recognized methodologies. In an exemplary embodiment, RNA can be purified from a mixture by extraction with a solvent or resin, precipitation, electrophoresis, chromatography, or a combination thereof. Alternatively, the RNA may be used with no or a minimum of purification to avoid losses due to sample processing. Alternatively, the single-stranded RNAs can also be prepared by enzymatic transcription from synthetic DNA templates or from DNA plasmids isolated from recombinant bacteria. Typically, phage RNA polymerases are used such as T7, T3 or SP6 RNA polymerase (Milligan and Uhlenbeck (1989) Methods Enzymol. 180:51-62). The RNA may be dried for storage or dissolved in an aqueous solution. The solution may contain buffers or salts to inhibit annealing, and/or promote stabilization of the single strands.

### III. Targets

In one embodiment, the target mRNA of the invention specifies the amino acid sequence of a cellular protein (e.g., a nuclear, cytoplasmic, transmembrane, or membrane-associated protein). In another embodiment, the target mRNA of the invention specifies the amino acid sequence of an extracellular protein (e.g., an extracellular matrix protein or secreted protein). As used herein, the phrase “specifies the amino acid sequence” of a protein means that the mRNA sequence is translated into the amino acid sequence according to the rules of the genetic code.

In a preferred aspect of the invention, the target mRNA molecule of the invention specifies the amino acid sequence of a mutant protein associated with a pathological condition. For example, the protein may be a gain-of-function (e.g., a dominant gain-of-function) mutant protein. In a preferred aspect, the mutant protein is associated with a disease or disorder which is correlated with expression of a particular allele of a gene, e.g., a dominant gain-of-function mutation. For example, such disorders may include amyotrophic lateral sclerosis, Huntington's disease, Alzheimer's disease, and Parkinson's disease. Thus, in one embodiment, the mutant protein is an allele-specific mutant protein, (e.g., an allele-specific dominant gain-of-function mutant protein).

A number of disorders have been linked to gain-of-function mutations, including well-known disorders such as amyotrophic lateral sclerosis (ALS), Huntington's disease (48-49), Alzheimer's disease, and Parkinson's disease. Gain-of-function mutations in the KIT receptor have been linked to a number of gastrointestinal stromal tumors (41-42). Naturally occurring mutations in G protein alpha subunits and in G protein-coupled receptors have been linked to a number of human diseases, including endocrine disorders (43). Germline loss of function mutations in the ubiquitously expressed Gs-alpha gene have been identified as the cause of generalized hormone resistance and dysmorphic features in the inherited disorder pseudohypoparathyroidism type Ia. Somatic gain-of-function mutations in Gs-alpha have been identified as the cause of the McCune-Albright syndrome, a sporadic disorder in which affected individuals have varying combinations of endocrine hyperfunction, café-au-lait skin pigmentation, and polyostotic fibrous dysplasia.

Gain-of-function mutations in the thyrotropin receptor (TSHR, a G-protein coupled receptor) are correlated with toxic follicular thyroid adenoma, a condition caused by excessive quantities of thyroid hormones (44). Gain-of-function mutations in TSH receptor genes have also been linked to hereditary toxic thyroid hyperplasia, another condition caused by excessive quantities of thyroid hormones (45). Mutations of the superoxide dismutase (SOD) gene have been linked to certain familial forms of ALS (46). Mutations in protein-tyrosine phosphatase, nonreceptor-type 11 (PTPN11) have been correlated with Noonan syndrome, an autosomal dominant disorder characterized by dysmorphic facial features, proportionate short stature and heart disease (47). Hereditary pancreatitis is associated with mutations in human cationic trypsinogen (50). And brachydactyly type B (BDB), an autosomal dominant disorder characterized by terminal deficiency of the fingers and toes, is believed to be associated with dominant gain-of-function mutation in ROR2, which encodes an orphan receptor tyrosine kinase. In the typical form of BDB, the thumbs and big toes are spared, sometimes with broadening or partial duplication (51). von Willebrand disease, particularly Type 2A and 2B, is another disease which may be associated with a dominant gain-of-function mutation (52). A dominant gain-of-function mutation has been described in p53 that results in oncogenic activation of that gene (53-54). In addition, Creutzfeldt-Jakob disease has been associated with a dominant gain-of-function mutation in the prion protein gene, the PRNP E200K mutation (55). Testotoxicosis is an autosomal dominant condition caused by a gain-of-function mutation in the LH receptor (56).

By inhibiting the expression of such proteins, e.g., allele-specific dominant gain-of-function mutant proteins, valuable information regarding the function of said proteins and therapeutic benefits which may be obtained from said inhibition may be obtained.

### IV. Pharmaceutical Compositions and Methods of Administration

The siRNA molecules of the invention can be incorporated into pharmaceutical compositions. Such compositions typically include the nucleic acid molecule and a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer. Such methods include those described in U.S. Pat. No. 6,468,798.

Systemic administration of an siRNA can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds (siRNAs) can also be prepared in the form of suppositories (e.g., with conventional suppository bases such as cocoa butter and other glycerides) or retention enemas for rectal delivery.

The compounds (siRNAs) can also be administered by transfection or infection using methods known in the art, including but not limited to the methods described in McCaffrey et al. (2002), Nature, 418(6893), 38-9 (hydrodynamic transfection); Xia et al. (2002), Nature Biotechnol., 20(10), 1006-10 (viral-mediated delivery); or Putnam (1996), Am. J. Health Syst. Pharm. 53(2), 151-160, erratum at Am. J. Health Syst. Pharm. 53(3), 325 (1996).

The compounds (siRNAs) can also be administered by any method suitable for administration of nucleic acid agents, such as a DNA vaccine. These methods include gene guns, bio injectors, and skin patches as well as needle-free methods such as the micro-particle DNA vaccine technology disclosed in U.S. Pat. No. 6,194,389, and the mammalian transdermal needle-free vaccination with powder-form vaccine as disclosed in U.S. Pat. No. 6,168,587. Additionally, intranasal delivery is possible, as described in, inter alia, Hamajima et al. (1998), Clin. Immunol. Immunopathol., 88(2), 205-10. Liposomes (e.g., as described in U.S. Pat. No. 6,472,375) and microencapsulation can also be used. Biodegradable targetable microparticle delivery systems can also be used (e.g., as described in U.S. Pat. No. 6,471,996).

In one embodiment, the siRNAs are prepared with carriers that will protect the siRNAs against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Dosage, toxicity and therapeutic efficacy of such siRNA compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of a nucleic acid molecule such as an siRNA (i.e., an effective dosage) depends on the nucleic acid selected. For instance, single dose amounts in the range of approximately 1:g to 1000 mg may be administered; in some embodiments, 10, 30, 100 or 1000:g may be administered. In some embodiments, 1-5 g of the compositions can be administered. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the siRNAs of the invention can include a single treatment or, can include a series of treatments.

The pharmaceutical compositions can be included in a container, pack, or dispenser together with instructions for administration.

### V. Methods of Treatment

The present invention provides for both prophylactic and therapeutic methods of treating a subject at risk of (or susceptible to) a disorder, or having a disorder, associated with expression of a particular allele of a gene, e.g., a dominant gain-of-function mutation. For example, such disorders may include amyotrophic lateral sclerosis, Huntington's disease, Alzheimer's disease, and Parkinson's disease. As used herein, the term “treatment” is defined as the application or administration of the siRNA compositions to a patient, or application or administration of a therapeutic composition including the siRNA compositions to an isolated tissue or cell line from a patient, who has a disease, a symptom of disease, or a predisposition toward a disease, with the purpose to cure, heal, alleviate, relieve, alter, remedy, ameliorate, improve, or affect the disease, the symptoms of disease, or the predisposition toward disease. The presence or predisposition to the disease can be confirmed by determining all or part of the genotype of the patient using routine methods, generally including that portion of the genotype of the patient that is known to be associated with a disease. The treatment can include administering siRNAs to one or more target sites on one or more target alleles. The mixture of different siRNAs may be administered together or sequentially, and the mixture may be varied over occasion.

With regards to both prophylactic and therapeutic methods of treatment, such treatments can be specifically tailored or modified, based on knowledge obtained from the field of genomics, particularly genomics technologies such as gene sequencing, statistical genetics, and gene expression analysis, as applied to a patient's genes. Thus, another aspect of the invention provides methods for tailoring an individual's prophylactic or therapeutic treatment with the siRNA compositions of the present invention according to that individual's genotype; e.g., by determining the exact sequence of relevant region of the patient's genome and designing, using the present methods, an siRNA molecule customized for that patient. This allows a clinician or physician to tailor prophylactic or therapeutic treatments to patients to enhance the effectiveness or efficacy of the present methods.

I. Prophylactic Methods

In one aspect, the invention provides a method for preventing in a subject, a disease or condition associated with an aberrant or unwanted target gene expression or activity, by administering to the subject a therapeutic agent (e.g., a siRNA modified as described herein). Subjects at risk for a disease which is caused or contributed to by aberrant or unwanted target gene expression or activity can be identified by, for example, any or a combination of diagnostic or prognostic assays as described herein. Administration of a prophylactic agent can occur prior to the manifestation of symptoms characteristic of the target gene aberrancy, such that a disease or disorder is prevented or, alternatively, delayed in its progression. Depending on the type of target gene aberrancy, for example, a target gene, target gene agonist or target gene antagonist agent can be used for treating the subject. The appropriate agent can be determined based on screening assays described herein.

2. Therapeutic Methods

Another aspect of the invention pertains to methods of modulating target gene expression, protein expression or activity for therapeutic purposes. Accordingly, in an exemplary embodiment, the modulatory method of the invention involves contacting a cell capable of expressing target gene with a therapeutic agent (e.g., a siRNA modified as described herein) that is specific for the target gene or protein (e.g., is specific for the mRNA encoded by said gene, e.g., allele, or specifying the amino acid sequence of said protein, e.g., dominant gain-of-function mutant protein) such that expression or one or more of the activities of target protein is modulated. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant or unwanted expression or activity of a target gene polypeptide or nucleic acid molecule. Inhibition of target gene activity is desirable in situations in which target gene is abnormally unregulated and/or in which decreased target gene activity is likely to have a beneficial effect.

3. Pharmacogenomics

The therapeutic agents (e.g., a siRNA) of the invention can be administered to individuals to treat (prophylactically or therapeutically) disorders associated with aberrant or unwanted target gene activity. In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer a therapeutic agent as well as tailoring the dosage and/or therapeutic regimen of treatment with a therapeutic agent.

Pharmacogenomics deals with clinically significant hereditary variations in the response to drugs due to altered drug disposition and abnormal action in affected persons. See, for example, Eichelbaum, M. et al. (1996) Clin. Exp. Pharmacol. Physiol. 23(10-11): 983-985 and Linder, M. W. et al. (1997) Clin. Chem. 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants.) Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten-million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach”, can be utilized to identify genes that predict drug response. According to this method, if a gene that encodes a drugs target is known (e.g., a target gene polypeptide of the present invention), all common variants of that gene can be fairly easily identified in the population and it can be determined if having one version of the gene versus another is associated with a particular drug response.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Alternatively, a method termed the “gene expression profiling”, can be utilized to identify genes that predict drug response. For example, the gene expression of an animal dosed with a therapeutic agent of the present invention can give an indication whether gene pathways related to toxicity have been turned on.

Information generated from more than one of the above pharmacogenomics approaches can be used to determine appropriate dosage and treatment regimens for prophylactic or therapeutic treatment an individual. This knowledge, when applied to dosing or drug selection, can avoid adverse reactions or therapeutic failure and thus enhance therapeutic or prophylactic efficiency when treating a subject with a therapeutic agent, as described herein.

Therapeutic agents can be tested in an appropriate animal model. For example, an siRNA (or expression vector or transgene encoding same) as described herein can be used in an animal model to determine the efficacy, toxicity, or side effects of treatment with said agent. Alternatively, a therapeutic agent can be used in an animal model to determine the mechanism of action of such an agent. For example, an agent can be used in an animal model to determine the efficacy, toxicity, or side effects of treatment with such an agent. Alternatively, an agent can be used in an animal model to determine the mechanism of action of such an agent.

The following materials, methods, and examples are illustrative only and not intended to be limiting.

### EXAMPLES

### Example 1

### Quantitative Analysis of RNAi Effects in HeLa Cells Transfected with U(5I) Modified Duplex siRNAs

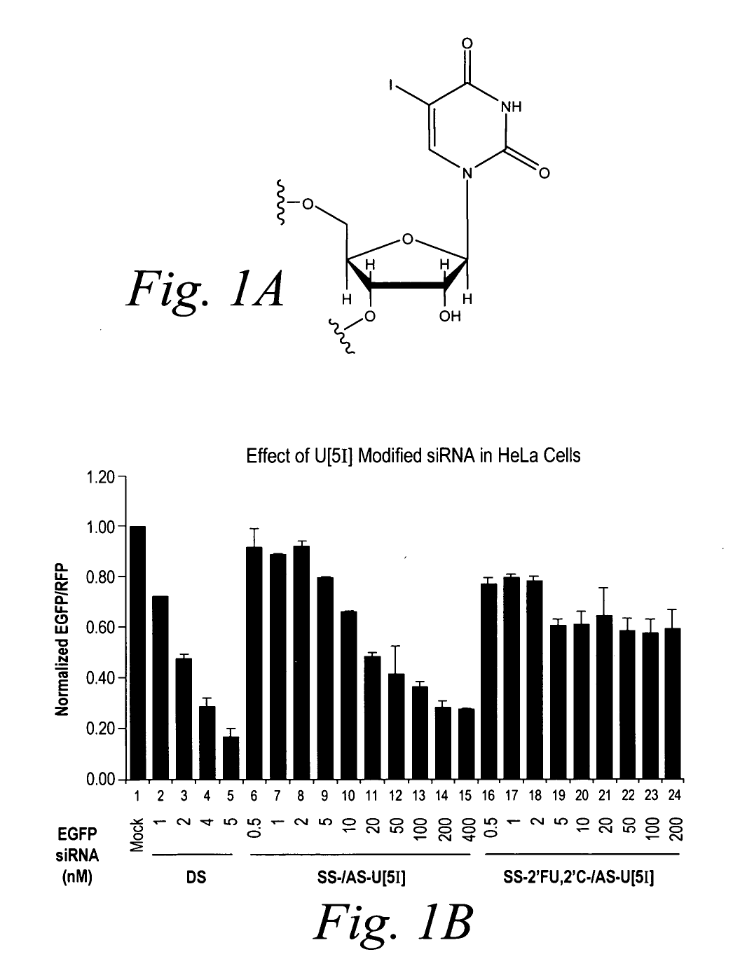

To determine whether a modified duplex siRNA can be used to selectively decrease expression of a particular allele, siRNAs having U(5I) modification were synthesized. Specifically, a reporter plasmid, pEGFP-C1 (Clontech) and control plasmid, pDsRed2-N1 (Clontech), and various amount of modified siRNA were cotransfected into HeLa cells using Lipofectamine™, as shown in FIG. 1B. Cells were harvested 42 hours after transfection. The fluorescence intensity of Green Fluorescent Protein (GFP) and Red Fluorescent Protein (RFP) in total cell lysates was detected by exciting at 488 and 568 nm, respectively. The fluorescence intensity ratio of target (GFP) to control (RFP) fluorophore was determined in the presence of modified siRNAs and normalized to the ratio observed in mock treated cells. Normalized ratios of less than 1.0 indicate specific RNA interference effects. FIG. 1B shows the results from cells treated with duplex siRNA with U(5I) modification in the antisense strand. Modified siRNA were formed by annealing U(5I) modified antisense strand with unmodified (ss/as-U(5I), bars 6-15) or with sense strand containing 2′-fluoro modification at uridine and cytidine base (ss-2′FU,FC/as-U(5I), bars 16-24). Modified and unmodified sense and antisense strands were obtained by custom order from Dharmacon (Lafayette, Colo.). For comparison, results from unmodified duplex siRNA (ds, bars 2-5)-treated cells are included. These results show that 5-lodo-uridine (U(5I)) modification is tolerated in the RNA interference pathway, i.e., does not interfere with gene-specific silencing. Additional siRNA can be used to achieve activity comparable to unmodified duplexes. Less than complete inhibition of expression of an allele can be useful when a partial decrease is sufficient to ameliorate the effects of expression of the targeted allele.

### Example 2

### Quantitative Analysis of RNAi Effects in HeLa Cells Transfected with DAP-modified Duplex siRNAs

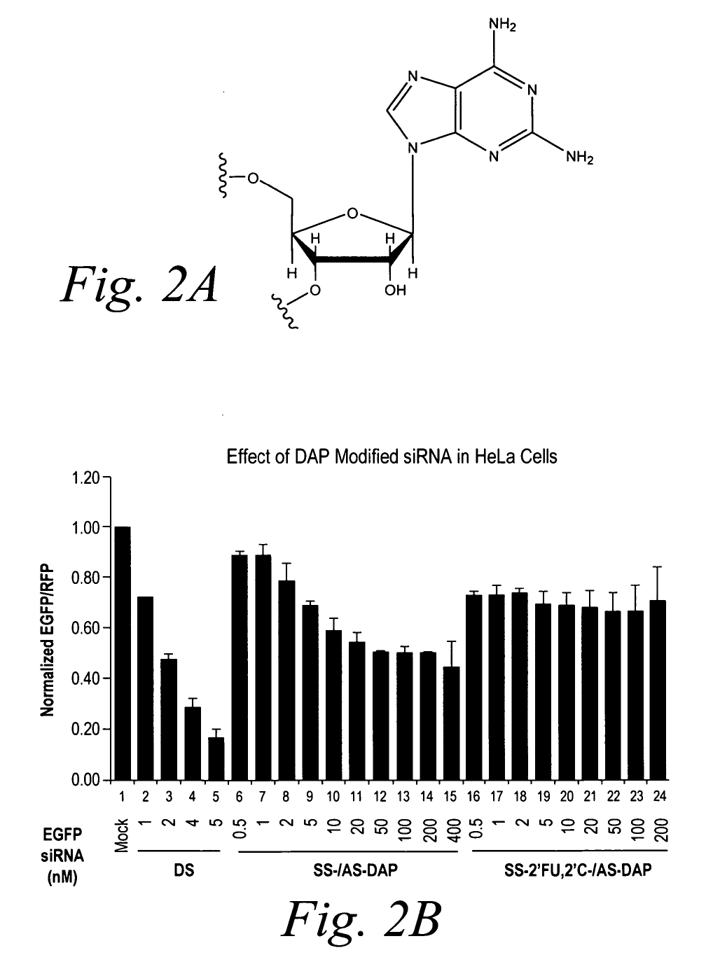

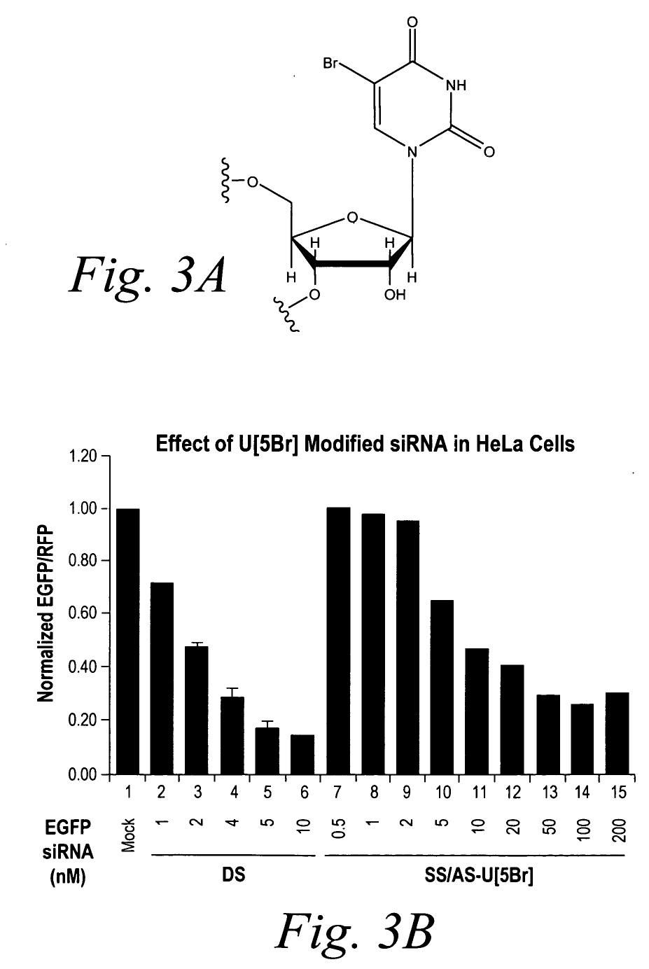

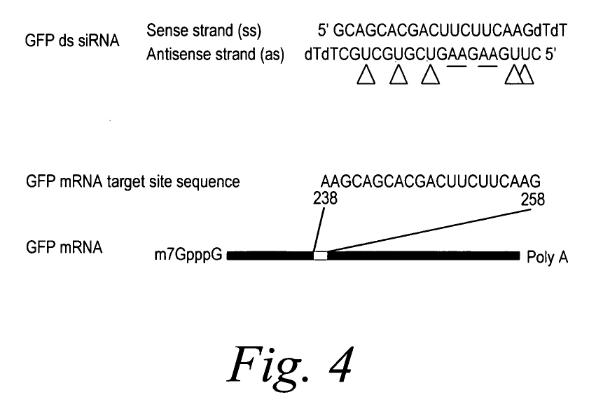

To determine whether a DAP-modified siRNA duplex can be used to decrease expression of a targeted sequence, pEGFP-C1 (as reporter) and pDsRed2-N1 (as control) plasmids and various amount of modified siRNA were cotransfected into HeLa cells using Lipofectamine™. The cells were harvested 42 hours after transfection. Fluorescence intensity of GFP and RFP in total cell lysates was detected by exciting at 488 and 568 nm, respectively. Fluorescence intensity ratio of target (GFP) to control (RFP) fluorophore was determined in the presence of modified siRNAs and normalized to the ratio observed in the mock-treated cells. Normalized ratios of less than 1.0 indicate specific RNA interference effects. The results from cells treated with duplex siRNA with DAP modification at antisense strand are shown in FIG. 2B. A residue of the antisense strand was replaced by DAP. Modified duplex siRNAs were formed by annealing DAP modified antisense strand with unmodified (ss/as-U(5I), bars 6-15) or with sense strand containing 2′-fluoro modification at uridine and cytidine base (ss-2′FU,FC/as-U(5I), bars 16-24). Modified and unmodified sense and antisense strands were obtained by custom order from Dharmacon (Lafayette, Colo.). For comparison, results from unmodified duplex siRNA (ds, bars 2-5)-treated cells are included. This result shows that DAP modification is tolerated in RNA interference pathway although 20 fold more of siRNA is need to get comparable activity to unmodified duplex.

### Example 3

### Quantitative Analysis of RNAi Effects in HeLa Cells Transfected with U(5Br) Modified Duplex siRNAs

To determine whether a U(5Br) modified duplex siRNA can be used to decrease expression of a targeted sequence, HeLa cells were cotransfected with pEGFP-C1 (as reporter) and pDsRed2-N1 (as control) plasmids and various amount of modified siRNA by Lipofectamine™. Cells were harvested at 42 hours post-transfection. Fluorescence intensity of GFP and RFP in total cell lysates (300:g/160:l) were detected by exciting at 488 and 568 nm, respectively. The fluorescence intensity ratio of target (GFP) to control (RFP) fluorophore was determined in the presence of modified siRNAs and normalized to the ratio observed in the mock treated cells. Normalized ratios less than 1.0 indicates specific RNA interference effects. Results from cells treated with duplex siRNA with U[5Br] modification at antisense strand are shown in FIG. 1B. Modified siRNA were formed by annealing U[5Br] modified antisense strand with unmodified sense strand (ss/as-U[5Br], bars 7-15). Modified and unmodified sense and antisense strands were obtained by custom order from Dharmacon (Lafayette, Colo.). For comparison, results from unmodified duplex siRNA (ds, bars 2-6)-treated cells are included. This result shows that 5-bromo-uridine (U[5Br]) modification is well-tolerated in RNA interference pathway and its activity is higher than 5-lodo-uridine (U[5I]) modification and comparable to unmodified duplex siRNA.

### References

1. Taylor, J. P., Hardy, J. & Fischbeck, K. H. Toxic proteins in neurodegenerative disease. Science 296, 1991-5. (2002).

2. Hutvagner, G. & Zamore, P. D. RNAi: nature abhors a double-strand. Curr Opin Genet Dev 12, 225-32. (2002).

3. Hannon, G. J. RNA interference. Nature 418, 244-51. (2002).

4. McManus, M. T. & Sharp, P. A. Gene silencing in mammals by small interfering RNAs. Nat Rev Genet 3, 737-47. (2002).

5. Zamore, P. D., Tuschl, T., Sharp, P. A. & Bartel, D. P. RNAi: double-stranded RNA directs the ATP-dependent cleavage of mRNA at 21 to 23 nucleotide intervals. Cell 101, 25-33. (2000).

6. Hamilton, A. J. & Baulcombe, D. C. A species of small antisense RNA in posttranscriptional gene silencing in plants. Science 286, 950-2. (1999).

7. Hammond, S. M., Bernstein, E., Beach, D. & Hannon, G. J. An RNA-directed nuclease mediates post-transcriptional gene silencing in Drosophila cells. Nature 404, 293-6. (2000).

8. Elbashir, S. M., Lendeckel, W. & Tuschl, T. RNA interference is mediated by 21- and 22-nucleotide RNAs. Genes Dev 15, 188-200. (2001).

9. Caplen, N. J., Parrish, S., Imani, F., Fire, A. & Morgan, R. A. Specific inhibition of gene expression by small double-stranded RNAs in invertebrate and vertebrate systems. Proc Natl Acad Sci USA 98, 9742-7. (2001).

10. Elbashir, S. M. et al. Duplexes of 21-nucleotide RNAs mediate RNA interference in cultured mammalian cells. Nature 411, 494-8. (2001).

11. Paul, C. P., Good, P. D., Winer, I. & Engelke, D. R. Effective expression of small interfering RNA in human cells. Nat Biotechnol 20, 505-8. (2002).

12. Lee, N. S. et al. Expression of small interfering RNAs targeted against HIV-1 rev transcripts in human cells. Nat Biotechnol 20, 500-5. (2002).

13. Paddison, P. J., Caudy, A. A., Bernstein, E., Hannon, G. J. & Conklin, D. S. Short hairpin RNAs (shRNAs) induce sequence-specific silencing in mammalian cells. Genes Dev 16, 948-58. (2002).

14. Sui, G. et al. A DNA vector-based RNAi technology to suppress gene expression in mammalian cells. Proc Natl Acad Sci USA 99, 5515-20. (2002).

15. Yu, J. Y., DeRuiter, S. L. & Turner, D. L. RNA interference by expression of short-interfering RNAs and hairpin RNAs in mammalian cells. Proc Natl Acad Sci USA 99, 6047-52. (2002).

16. McManus, M. T., Petersen, C. P., Haines, B. B., Chen, J. & Sharp, P. A. Gene silencing using micro-RNA designed hairpins. Rna 8, 842-50. (2002).

17. Zeng, Y., Wagner, E. J. & Cullen, B. R. Both natural and designed micro RNAs can inhibit the expression of cognate mRNAs when expressed in human cells. Mol Cell 9, 1327-33. (2002).

18. Brummelkamp, T. R., Bernards, R. & Agami, R. A system for stable expression of short interfering RNAs in mammalian cells. Science 296, 550-3. (2002).

19. Miyagishi, M. & Taira, K. U6 promoter-driven siRNAs with four uridine 3′ overhangs efficiently suppress targeted gene expression in mammalian cells. Nat Biotechnol 20, 497-500. (2002).

20. Jacque, J. M., Triques, K. & Stevenson, M. Modulation of HIV-1 replication by RNA interference. Nature 418, 435-8. (2002).

21. Cleveland, D. W. & Rothstein, J. D. From Charcot to Lou Gehrig: deciphering selective motor neuron death in ALS. Nat Rev Neurosci 2, 806-19. (2001).

22. Matzuk, M. M., Dionne, L., Guo, Q., Kumar, T. R. & Lebovitz, R. M. Ovarian function in superoxide dismutase 1 and 2 knockout mice. Endocrinology 139, 4008-11. (1998).

23. Shefner, J. M. et al. Mice lacking cytosolic copper/zinc superoxide dismutase display a distinctive motor axonopathy. Neurology 53, 1239-46. (1999).

24. McFadden, S. L., Ding, D. & Salvi, R. Anatomical, metabolic and genetic aspects of age-related hearing loss in mice. Audiology 40, 313-21. (2001).

25. Flood, D. G. et al. Hindlimb motor neurons require Cu/Zn superoxide dismutase for maintenance of neuromuscular junctions. Am J Pathol 155, 663-72. (1999).

26. Matz, P. G., Copin, J. C. & Chan, P. H. Cell death after exposure to subarachnoid hemolysate correlates inversely with expression of CuZn-superoxide dismutase. Stroke 31, 2450-9. (2000).

27. Kondo, T. et al. Reduction of CuZn-Superoxide Dismutase Activity Exacerbates Neuronal Cell Injury and Edema Formation after Transient Focal Cerebral Ischemia. Journal of Neuroscience 17, 4180-9 (1997).

28. Kawase, M. et al. Exacerbation of delayed cell injury after transient global ischemia in mutant mice with CuZn superoxide dismutase deficiency. Stroke 30, 1962-8. (1999).

29. Behndig, A., Karlsson, K., Reaume, A. G., Sentman, M. L. & Marklund, S. L. In vitro photochemical cataract in mice lacking copper-zinc superoxide dismutase. Free Radic Biol Med 31, 738-44. (2001).

30. Boutla, A., Delidakis, C., Livadaras, I., Tsagris, M. & Tabler, M. Short 5′-phosphorylated double-stranded RNAs induce RNA interference in Drosophila. Curr Biol 11, 1776-80. (2001).

31. Brummelkamp, T., Bernards, R. & Agami, R. Stable suppression of tumorigenicity by virus-mediated RNA interference. Cancer Cell 2, 243. (2002).

32. Elbashir, S. M., Martinez, J., Patkaniowska, A., Lendeckel, W. & Tuschl, T. Functional anatomy of siRNAs for mediating efficient RNAi in Drosophila melanogaster embryo lysate. Embo J 20, 6877-88. (2001).

33. Caplen, N. J. et al. Rescue of polyglutamine-mediated cytotoxicity by double-stranded RNA-mediated RNA interference. Hum Mol Genet 11, 175-84. (2002).

34. Xia, H., Mao, Q., Paulson, H. L. & Davidson, B. L. siRNA-mediated gene silencing in vitro and in vivo. Nat Biotechnol 20, 1006-10. (2002).

35. Lipardi, C., Wei, Q. & Paterson, B. M. RNAi as random degradative PCR: siRNA primers convert mRNA into dsRNAs that are degraded to generate new siRNAs. Cell 107, 297-307. (2001).

36. Schwarz, D. S., Hutvagner, G., Haley, B. & Zamore, P. D. Evidence that siRNAs function as guides, not primers, in the Drosophila and human RNAi pathways. Molecular Cell 10, 537-548 (2002).

37. Chiu, Y.-L. & Rana, T. M. RNAi in human cells: basic structural and functional features of small interfering RNA. Molecular Cell 10, 549-561 (2002).

38. Tuschl, T., Zamore, P. D., Lehmann, R., Bartel, D. P. & Sharp, P. A. Targeted mRNA degradation by double-stranded RNA in vitro. Genes Dev 13, 3191-7 (1999).

39. Devroe, E. & Silver, P. A. Retrovirus-delivered siRNA. BMC Biotechnol 2, 15 (2002).

40. Nykanen, A., Haley, B. & Zamore, P. D. ATP requirements and small interfering RNA structure in the RNA interference pathway. Cell 107, 309-21 (2001).

41. Hirota S, Isozaki K, Moriyama Y, Hashimoto K, Nishida T, Ishiguro S, Kawano K, Hanada M, Kurata A, Takeda M, Muhammad Tunio G, Matsuzawa Y, Kanakura Y, Shinomura Y, Kitamura Y. Gain-of-function mutations of c-kit in human gastrointestinal stromal tumors. Science, 279(5350):577-80 (1998).

42 Hirota S, Nishida T, Isozaki K, Taniguchi M, Nakamura J, Okazaki T, Kitamura Y. Gain-of-function mutation at the extracellular domain of KIT in gastrointestinal stromal tumours. J. Pathol., 193(4):505-10 (2001).

43. Lania A, Mantovani G, and Spada A. G protein mutations in endocrine diseases. Eur. J. Endocrinol., 145(5):543-59 (2001).

44. Kopp P. The TSH receptor and its role in thyroid disease. Cell Mol Life Sci., 58(9):1301-22 (2001).

45. Puttaparthi K, Gitomer W L, Krishnan U, Son M, Rajendran B, Elliott J L. Disease progression in a transgenic model of familial amyotrophic lateral sclerosis is dependent on both neuronal and non-neuronal zinc binding proteins. J. Neurosci. 22(20):8790-6 (2002).

46. Duprez L, Parma J, Van Sande J, Rodien P, Sabine C, Abramowicz M, Dumont J E, Vassart G. Pathology of the TSH receptor. J Pediatr Endocrinol Metab. 12 Suppl 1:295-302 (1999).

47. Kosaki K, Suzuki T, Muroya K, Hasegawa T, Sato S, Matsuo N, Kosaki R, Nagai T, Hasegawa Y, Ogata T. PTPN11 (protein-tyrosine phosphatase, nonreceptor-type 11) mutations in seven Japanese patients with Noonan syndrome. J. Clin. Endocrinol. Metab. 87(8):3529-33 (2002).

48. Ho L W, Carmichael J, Swartz J, Wyttenbach A, Rankin J, Rubinsztein D C. The molecular biology of Huntington's disease. Psychol. Med. 31(1):3-14 (2001).

49. Zuccato C, Ciammola A, Rigamonti D, Leavitt B R, Goffredo D, Conti L, MacDonald M E, Friedlander R M, Silani V, Hayden M R, Timmusk T, Sipione S, Cattaneo E. Loss of huntingtin-mediated BDNF gene transcription in Huntington's disease. Science. 293(5529):493-8 (2001).

50. Sahin-Toth M, and Toth M. Gain-of-function mutations associated with hereditary pancreatitis enhance autoactivation of human cationic trypsinogen. Biochem. Biophys. Res Commun. 278(2):286-9 (2000).

51. Oldridge M, Fortuna AM, Maringa M, Propping P, Mansour S, Pollitt C, DeChiara T M, Kimble R B, Valenzuela D M, Yancopoulos G D, Wilkie A O. Dominant mutations in ROR2, encoding an orphan receptor tyrosine kinase, cause brachydactyly type B. Nat Genet. 24(3):275-8 (2000).

52. Fressinaud E, Mazurier C, Meyer D. Molecular genetics of type 2 von Willebrand disease. Int J Hematol. 75(1):9-18 (2002).

53. Hixon M L, Flores A, Wagner M, Gualberto A. Gain-of-function properties of mutant p53 proteins at the mitotic spindle cell cycle checkpoint. Histol. Histopathol. 15(2):551-6 (2000).

54. Gualberto A, Aldape K, Kozakiewicz K, Tlsty T D. An oncogenic form of p53 confers a dominant, gain-of-function phenotype that disrupts spindle checkpoint control. Proc. Natl. Acad. Sci. USA. 95(9):5166-71 (1998).

55. Simon E S, Kahana E, Chapman J, Treves T A, Gabizon R. Rosenmann H, Zilber N, Korczyn A D. Creutzfeldt-Jakob disease profile in patients homozygous for the PRNP E200K mutation. Ann. Neurol. 47(2):257-60 (2000).

56. Muller J, Gondos B, Kosugi S, Mori T, Shenker A. Severe testotoxicosis phenotype associated with Asp578—>Tyr mutation of the lutrophin/choriogonadotrophin receptor gene. J Med Genet. 35(4):340-1 (1998).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A small interfering RNA (siRNA) comprising at least one modified base, wherein the modified base is capable of enhancing single nucleotide discrimination between a first target having 1, 2, 3 or more mutations relative to a second target.

2. A small interfering RNA (siRNA) capable of single nucleotide discrimination between a first and second allele, the first allele having 1, 2, 3 or more mutations relative to the second allele, wherein the siRNA comprises at least one modified base capable of enhancing binding interactions between the siRNA and mRNA encoded by the first allele when compared with binding interactions between the siRNA and mRNA encoded by the second allele.

3. A small interfering RNA (siRNA) comprising a sense strand and an antisense strand, wherein the sense strand comprises a sequence homologous to a region of a mutant allele encoding a gain-of-function mutant protein, said region comprising one or more point mutations, and wherein the antisense strand comprises a sequence comprising one or more modified bases positioned opposite the point mutations, such that the siRNA directs allele-specific cleavage of a mRNA encoded by the mutant allele.

4. The siRNA of any one of claims 1-3, wherein the modified base is selected from the group consisting of 5-bromo-uridine, 5-bromo-cytidine, 5-iodo-uridine, 5-iodo-cytidine, 2-amino-purine, 2-amino-allyl-purine, 6-amino-purine, 6-amino-allyl-purine, 2,6-diaminopurine and 6-amino-8-bromo-purine.

5. The siRNA of claim 4, wherein the modified base is 5-bromo-uridine or 5-iodo-uridine.

6. The siRNA of claim 5, wherein the point mutation is an adenine.

7. The siRNA of claim 4, wherein the modified base is 2,6-diaminopurine.

8. The siRNA of claim 7, wherein the point mutation is a thymine.

9. The siRNAi of claim 3, which targets an allelic point mutation within a gene correlated with a disorder selected from the group consisting of amyotrophic lateral sclerosis, Huntington's disease, Alzheimer's disease, and Parkinson's disease.

10. The siRNA of any one of claims 1-3, which is between about 10 and 50 residues in length.

11. The siRNA of any one of claims 1-3, which is between about 15 and 45 residues in length.

12. The siRNA of any one of claims 1-3, which is between about 20 and 40 residues in length.

13. The siRNA of any one of claims 1-3, which is between about 18-25 residues in length.

14. A therapeutic composition, comprising the siRNA of any one of claims 1-3 and a pharmaceutically acceptable carrier.

15. A host cell comprising the siRNAi of any one claims 1-3.

16. The host cell of claim 15, which is mammalian cell.

17. The host cell of claim 15, which is a human cell.

18. A method of selectively targeting in a cell a first allele having 1, 2, 3 or more mutations relative to a second allele, the method comprising contacting the cell with an siRNA according to any one of claims 1-3 having a sequence specific for the first allele, such that the first allele is selectively targeted.

19. A method of inhibiting expression of a target allele in a cell comprising at least two different alleles of a gene, the method comprising introducing into the cell an siRNA according to any one of claims 1-3 having a sequence specific for the target allele, said siRNA being introduced in an amount sufficient for degradation of a mRNA encoded by the target allele to occur, thereby inhibiting expression of the target allele.

20. The method of claim 19, wherein the target allele is correlated with a disease or disorder associated with a dominant gain-of-function mutation.

21. The method of claim 20, wherein the disease or disorder is chosen from the group consisting of amyotrophic lateral sclerosis, Huntington's disease, Alzheimer's disease, and Parkinson's disease.

22. The method of claim 19, wherein the expression is inhibited by at least 10%.

23. A cell obtained by the methods of claim 19.

24. A cell of claim 23, which is of mammalian origin.

25. A cell of claim 24, which is of human origin.

26. A cell of claim 24, which is an embryonic stem cell.

27. A method of activating allele-specific RNA interference (RNAi) in an organism comprising at least two different alleles of a gene, the method comprising administering to the organism the siRNA of any one of claims 1-3 having a sequence specific for the target allele, said siRNA being administered in an amount sufficient for degradation of the target allele mRNA to occur, thereby activating allele-specific RNAi in the organism.

28. The method of claim 27, wherein the target allele is correlated with a disease or disorder associated with a dominant gain-of-function mutation.

29. The method of claim 28, wherein the disease or disorder is chosen from the group consisting of amyotrophic lateral sclerosis, Huntington's disease, Alzheimer's disease, and Parkinson's disease.

30. The organism obtained by the method of claim 27.

31. A method of treating a subject having a disease or disorder correlated with the presence of a dominant gain-of-function mutant allele, the method comprising administering to the subject an siRNA of any one of claims 1-3 having a sequence specific for the mutant allele, said siRNA being administered in an amount sufficient for degradation of a mRNA encoded by the mutant allele to occur, thereby treating the subject.

32. The method of claim 31, wherein the disease or disorder is chosen from the group consisting of amyotrophic lateral sclerosis, Huntington's disease, Alzheimer's disease, and Parkinson's disease.

33. The method of claim 31, wherein the siRNA is targeted to the gain-of-function mutation.

34. The method of claim 31, wherein the mutant allele comprises one or more point mutations.
